(19) United States
(12) Patent Application Publication
(10) Pub. No.: US 2026/0242765 A1
(43) Pub. Date: Aug. 20, 2026

(54) NOVEL RECOMBINANT LYSINE N-TERMINAL PROTEASE AND PREPARATION METHOD THEREFOR

(71) Applicant: ACADEMY OF MILITARY MEDICAL SCIENCES, Beijing (CN)

(72) Inventors: Ping XU, Beijing (CN); Jiaxing DAI, Beijing (CN); Jiahui SHI, Beijing (CN); Lei CHANG, Beijing (CN)

(21) Appl. No.: 19/160,798

(22) PCT Filed: Jan. 8, 2024
(86) PCT No.: PCT/CN2024/071067

§ 371 Date: Aug. 28, 2025

(30) Foreign Application Priority Data
Feb. 28, 2023 (CN) 202310179253.9

(51) Int. Cl. C12N 9/58; C12N 9/52; C12N 15/70

(52) U.S. Cl. CPC C12N 9/58; C12N 9/52; C12N 15/70; C12Y 304/2402

(57) ABSTRACT

Provided are a novel recombinant protein for lysine N-terminal protease and a preparation method therefor. A gene for encoding the recombinant protease for lysine N-terminal protease and a protein thereof are screened and identified, wherein the protease has high activity, good specificity and high tolerance to high temperature as well as high concentration of SDS, and has an amino acid sequence shown in SEQ ID NO. 2. The recombinant protein for lysine N-terminal protease has high enzyme activity and specificity.

### FIELD OF THE INVENTION

The present invention belongs to the field of genetic engineering and relates to a recombinant lysine N-terminal protease and its preparation method.

### BACKGROUND OF THE INVENTION

The proteome is the identification and quantification of all proteins in a cell, tissue or organ, including the gene products as well as post-translational modification. Studying the abundance, function, interaction, localization, and regulation of proteins in cells is crucial to our understanding of the mysteries of life. In the most common shotgun proteomics research currently, the identification of proteins and post-translational modifications requires enzymatic digestion of proteins into peptide fragments, followed by mass spectrometry detection to obtain the primary spectra of peptide precursor ions and their secondary spectra generated from fragmentation in the mass spectrometer. The experimental spectra are then matched with the theoretical spectra derived from the existing proteins and genome databases (known as database searching) to infer the amino acid sequence of the peptide fragment to be tested. Among these steps, enzymatic digestion of a protein into peptide fragments is the core step in current proteomics research.

Trypsin, as the most commonly used protease in proteomics research, has been widely used in practice. On the one hand, it exhibits high cleavage efficiency and specificity, capable of specifically cleaving and hydrolyzing protein substrates at the carboxyl terminus of lysine and arginine residues (however, hydrolysis does not occur when the amino acid following lysine or arginine is proline). On the other hand, short peptides that terminate with arginine or lysine formed by enzymatic cleavage are more suitable for ionization and fragmentation of peptide fragments, forming stronger y and weaker b series ion pairs, which are suitable for separation and identification. However, since many of the peptide fragments formed by trypsin cleavage cannot be identified by mass spectrometry, resulting in overall low coverage [Swaney D L et al., Value of using multiple proteases for large-scale mass spectrometry-based proteomics. J Proteome Res, 2010, 9 (3): 1323-9]. In addition, trypsin is prone to miscleavage at post-translationally modified sites (such as methylation, acylation, ubiquitination, etc.), resulting in increased peptide lengths and reduced ionization efficiency, which affects the identification of modification sites and is detrimental to the functional analysis of important proteins.

The discovery, introduction, and combination of new proteases can significantly improve the coverage of proteome sequence and enhance the efficiency of identifying post-translational modification sites. Lysine N-terminal protease, also known as Peptidyl-Lys metalloendopeptidase (MEP, EC: 3.4.24.20), belongs to the Aspzincin family within the Zinc metalloproteases family, and can specifically cleave the N-terminal lysine of protein substrates. Currently, there are three main types of Peptidyl-Lys metalloendopeptidase reported, namely AmMEP from Armillaria mellea, GfMEP from Grifolafrondosa and PoMEP from Pleurotus ostreatus. Currently, only natural GfMEP from Grifolafrondosa (Zhao Mingzhi et al., Peptidyl-Lys metalloendopeptidase (Lys-N) purified from dry fruit of Grifolafrondosa demonstrates “mirror” digestion property with lysyl endopeptidase (Lys-C), Rapid Commun Mass Spectrom, 2020, 34 (2): e8573.doi: 10.1002/rcm.8573) and a 25 kDa recombinant LysN with undisclosed gene sequence from U-Protein Express (http://uprotein.ebiomall.cn/) are commercially available. AmMEP from Armillaria mellea has been successfully expressed in Pichia pastoris through eukaryotic recombinant expression, yielding active AmMEP, but the yield is extremely low and cannot meet industrial production requirements (Ødum A S et al., Heterologous expression of peptidyl-Lys metallopeptidase of Armillaria mellea and mutagenic analysis of the recombinant peptidase. J Biochem, 2016, 159 (4): 461-70). Similar to GfMEP and PoMEP, there have been no successful reports to date of recombinant expression using a prokaryotic system to obtain active AmMEP. To screen and identify specific and efficient lysine N-terminal proteases, Rawlings et al. conducted a comprehensive homology analysis of sequences stored in GenBank using existing lysine N-terminal proteases and lysine/arginine N-terminal protease motifs (zinc-binding motif HEXXH and metal-coordinating motif responsible for catalysis GTXDXXYG), thereby identifying thousands of potential proteases from members of the MEROPS superfamily M10, M12, M35, M43, M54, M57, M66, M72, M80, M84, and M97 (Rawlings et al., The MEROPS database of proteolytic enzymes, their substrates and inhibitors in 2017 and a comparison with peptidases in the PANTHER database. Nucleic Acids Res, 2018, 46: D624-D632, doi: 10.1093/nar/gkx1134). Wilson et al. further excluded numerous small proteins, existing lysine N-terminal proteases and lysine/arginine N-terminal proteases, and transmembrane domain structures from this long protein list, yielding 28 candidate gene sequences (Wilson et al., Tryp-N: A thermostable protease for the production of N-terminal argininyl and lysinyl peptides, J Proteome Res, 2020, 19 (4): 1459-1469). Using an in vitro transcription and translation system, Wilson et al. identified the site-specific and highly active lysine and arginine N-terminal protease Tryp-N, and conducted gene recombination, expression, and purification studies. The function of genes tested in this study that may encode lysine N-terminal protease have laid the groundwork for future research.

### SUMMARY OF THE INVENTION

One object of the present invention is to provide a novel recombinant lysine N-terminal protease.

The second object of the present invention is to provide a nucleic acid molecule encoding the recombinant lysine N-terminal protease.

The third object of the present invention is to provide an expression vector.

The fourth object of the present invention is to provide a host cell.

The fifth object of the present invention is to provide a method for preparing the recombinant lysine N-terminal protease.

The sixth object of the present invention is to provide the recombinant lysine N-terminal protease prepared according to the method.

The technical problem to be solved by the present invention is the identification of a more efficient peptidyl-lysine metalloendopeptidase. Using molecular biology techniques, Escherichia coli (E. coli) is employed as the host cell to construct an expression vector for Peptidyl-Lys metalloendopeptidase (MEP) (LysN), and the protein purification methods were optimized to obtain a recombinant lysine N-terminal protease (SL-LysN) with high purity, high activity and high specificity.

According to the first aspect of the present invention, a novel recombinant lysine N-terminal protease (SL-LysN) is provided, which is derived from the recombinant lysine N-terminal protease of Shewanella loihica and subjected to conservative site-directed mutagenesis, having the amino acid sequence shown in SEQ ID NO. 2.

According to the second aspect of the present invention, the gene encoding the above-mentioned recombinant lysine N-terminal protease is subjected to codon optimization to obtain a nucleic acid suitable for expression in Escherichia coli, wherein the nucleic acid has the nucleotide sequence shown in SEQ ID NO. 3.

According to the third aspect of the present invention, the nucleic acid obtained by the above-mentioned codon optimization is operably inserted into the pET28a vector to construct an expression vector suitable for Escherichia coli.

According to the fourth aspect of the present invention, the above-mentioned expression vector is introduced into an E. coli host cell. The host cell can express the recombinant lysine N-terminal protease under expression conditions.

According to the fifth aspect of the present invention, a method for preparing the recombinant lysine N-terminal protease comprises the following steps:
(a) operably linking the gene encoding the recombinant lysine N-terminal protease shown in SEQ ID NO. 3 to an expression vector, and introducing the expression vector into a host cell;
(b) culturing the host cell as described above under expression conditions to express the recombinant lysine N-terminal protease; and
(c) isolating and purifying the recombinant lysine N-terminal protease from (b).

More preferably, the method for preparing the novel recombinant lysine N-terminal protease provided by the present invention comprises:
(1) introducing the gene encoding the recombinant lysine N-terminal protease having the amino acid sequence shown in SEQ ID NO. 2 into recipient E. coli cells to obtain recombinant E. coli cells;
(2) inducing expression in the recombinant E. coli cells, collecting the recombinant E. coli cells; and lysing the recombinant E. coli cells to obtain inclusion bodies;
(3) denaturing the inclusion bodies to obtain a denatured sample;
(4) renaturing the denatured sample to obtain a renatured sample;
(5) activating the renatured sample to obtain crude recombinant lysine N-terminal protease with activity; and
(6) purifying the crude recombinant lysine N-terminal protease to obtain purified recombinant lysine N-terminal protease.

In step (3), the denaturation is performed in a denaturing buffer, which has a pH value of 8.5-9.5, specifically 9.0; the denaturing buffer comprises a solvent and solutes, where the solvent is water, and the solutes are tris(hydroxymethyl)amino-methane, dithiothreitol, and urea; the concentration of tris(hydroxymethyl)amino-methane in the denaturing buffer is 20-50 mM, specifically 20 mM; the concentration of dithiothreitol in the denaturing buffer is 10-20 mM, specifically 10 mM; and the concentration of urea in the denaturing buffer is 7.5-8.5 M, specifically 8 M.

In step (4), the renaturation is performed in a renaturing buffer, which has a pH value of 8.0-10.0, specifically 9.0; the renaturing buffer comprises a solvent and solutes, where the solvent is water, and the solutes are tris(hydroxymethyl)amino-methane, cystine, cysteine, glycine, arginine hydrochloride and glycerol; the concentration of tris(hydroxymethyl)aminomethane in the renaturing buffer is 10-50 mM, specifically 20 mM; the concentration of cystine in the renaturing buffer is 0.5-1.5 mM, specifically 1 mM; the concentration of cysteine in the renaturing buffer is 3-5 mM, specifically 5 mM; the concentration of glycine in the renaturing buffer is 0.1-0.4 M, specifically 0.3 M; the concentration of arginine hydrochloride in the renaturing buffer is 0.1-0.2 M, specifically 0.1 M; the concentration of glycerol in the renaturing buffer is 2-5%, specifically 2.5%.

In step (5), the activation comprises adding ZnSO₄ to the renatured sample to a concentration of 1 mM, followed by standing at 16° C. for 1-6 h, then adding disodium ethylenediaminetetraacetate to a final concentration of 2 mM to terminate the activation, and adjusting the pH of the sample to 4.0-5.0, specifically 4.0.

In step (6), the purification comprises sequential cation exchange chromatography, ultrafiltration concentration, and gel filtration chromatography.

The cation exchange chromatography uses SP Sepharose Fast Flow (SP-high flow rate agarose gel) or SP Sepharose High Performance (SP-high resolution agarose gel) as the chromatographic medium; the ultrafiltration concentration is an ultrafiltration concentration with a molecular weight cut-off of 10 kD; the gel filtration chromatography uses Superdex 75 Prep Grade as the chromatographic medium;

wherein, prior to gel filtration chromatography, the crude recombinant lysine N-terminal protease solution may undergo a freeze-thaw step at −20° C. or −80° C. for 12-18 h (overnight, specifically, such as 16 h).

In the present invention, the gene encoding the recombinant lysine N-terminal protease may specifically be the following nucleic acid molecule a) or b):
a) a DNA molecule having the coding sequence shown in SEQ ID NO. 3 in the sequence listing;
b) a DNA molecule that hybridizes with the DNA molecule defined in a) under stringent conditions and encodes the recombinant lysine N-terminal protease shown in SEQ ID NO. 2 in the sequence listing.

The above-mentioned recombinant lysine N-terminal protease gene can be easily mutated by a person skilled in the art using known methods, such as directed evolution and point mutagenesis, to mutate the gene sequence encoding the lysine N-terminal protease. Those artificially modified sequences that have 75% or higher identity with the gene sequence encoding the recombinant lysine N-terminal protease and have the same function are derived from the nucleotide sequence of the present invention and are equivalent to the sequence of the present invention.

The term “identity” used herein refers to sequence similarity with natural nucleic acid sequence. “Identity” comprises nucleotide sequences that have 75% or higher, or 85% or higher, or 90% or higher, or 95% or higher identity with the DNA molecule shown in SEQ ID NO. 3 of the present invention. Identity can be evaluated by visual inspection or computer software. Using computer software, the identity between two or more sequences can be expressed as a percentage (%), which can be used to evaluate the identity between the relevant sequences.

The stringent conditions are as follows: hybridization at 68° C. in a solution of 2×SSC and 0.1% SDS, followed by two washes of the membrane, each for 5 min; then hybridization at 68° C. in a solution of 0.5×SSC and 0.1% SDS, followed by two washes of the membrane, each for 15 min.

In the present invention, the gene encoding the recombinant lysine N-terminal protease shown in SEQ ID NO. 1 in the sequence listing is a gene that has not undergone codon optimization; the optimization involves replacing the codons with those preferred (highly used) by E. coli without altering the amino acid sequence of the wild-type lysine N-terminal protease. The optimization also includes other modifications to the gene sequence of the lysine N-terminal protease to suit expression in Escherichia coli. The term “preferred codon” used in the present invention has the meaning known in the art, also referred to as codon preference, which refers to the tendency of certain organisms to preferentially use certain synonymous triplet codons (i.e., codons encoding the same amino acid).

In step (1), the gene encoding the recombinant lysine N-terminal protease is introduced into the recipient E. coli cells via a recombinant expression vector. More specifically, the recombinant expression vector is a recombinant plasmid obtained by replacing the DNA fragment between the Nco I/Xho I recognition sequences of the pET28a vector with the DNA fragment shown at the first position of SEQ ID NO. 3 in the sequence listing.

In the present invention, the E. coli may specifically be E. coli BL21 (DE3).

In the method, the elution procedure for cation exchange chromatography may specifically be as follows: 1) performing NaCl linear gradient elution using a mixture of equilibration buffer and elution buffer for a total of 6 column volumes, wherein the concentration of NaCl in the mixture increases linearly from 0 M to 0.6 M over the 6 column volumes; 2) performing elution using the elution buffer for a total of 4 column volumes, wherein the concentration of NaCl in the elution buffer is 1 M over the 4 column volumes. The equilibration buffer has a pH value of 4.0, and comprises a solvent and a solute. The solute is EDTA-2Na, and the concentration of the EDTA-2Na may be 0.5-2 mM, specifically 1 mM; the pH value of the solvent is 4.0, and the acetate ion concentration may be 10-50 mM, specifically be 25 mM acetic acid-sodium acetate buffer; the elution buffer has pH value of 4.0, and comprises a solvent and solutes; the solutes are NaCl and EDTA-2Na, and the concentration of NaCl is 1M, the concentration of the EDTA-2Na may be 0.5-2 mM, specifically 1 mM; the pH value of the solvent is 4.0, the acetate ion concentration may be 10-50 mM, specifically be 25 mM acetic acid-sodium acetate buffer; the acetic acid-sodium acetate buffer has pH value of 4.0, and comprises a solvent and solutes; the solvent is water; the solutes are glacial acetic acid and sodium acetate, and the acetate ion concentration may be 10-50 mM, specifically 25 mM.

In one embodiment of the present invention, the column volume of the chromatography column used for cation exchange chromatography is 60 mL. The method further includes a step of equilibrating the column with the equilibration buffer before and after loading.

In the method, the column length-to-inner diameter ratio of the gel filtration chromatography is 300:13 (e.g., 60 cm: 2.6 cm). The pH of the elution buffer used in gel filtration chromatography is 3-5. The elution buffer comprises a solvent and solutes; the solvent is water and the solutes are glacial acetic acid, EDTA-2Na, and sodium acetate. The concentration of EDTA-2Na may be 0.5-2 mM, specifically 1 mM; the concentration of the acetate ion may be 10-50 mM, specifically 25 mM.

In one embodiment of the present invention, the gel filtration chromatography is performed using an “XK 26/600 column with a column height of 600 mm”, produced by GE Healthcare. Prior to loading, the method further comprises a step of equilibrating the chromatography column using the elution buffer employed in the gel filtration chromatography. The loading volume during gel filtration chromatography is 5 mL, with a chromatography flow rate of 6 mL/min.

In the method, during cation exchange chromatography and gel filtration chromatography, the elution peaks corresponding to the target band appears at a position of about 20 kDa as determined by SDS-PAGE electrophoresis are collected for subsequent operations.

In the method, the ultrafiltration concentration refers to ultrafiltration concentration with a molecular weight cut-off of 10 kD.

In step (2) of the method, the induction of expression in the recombinant E. coli cells comprises adding IPTG to the culture system containing the recombinant E. coli to a final concentration of 0.7-1.2 mM, followed by induction at 25° C. for 9 h.

More specifically, step (2) comprises: inoculating the recombinant E. coli cells into a fermentation medium (initial OD₆₀₀ is 0.01); first culturing under condition 1 to obtain fermentation broth 1; then adding a feed supplement to the fermentation broth 1, and culturing under condition 1 to obtain fermentation broth 2; then adding a feed supplement to the fermentation broth 2, and culturing under condition 2, and then adding IPTG to a final concentration of 1 mM, inducing at 25° C. for 9 h to obtain fermentation broth 3; collecting the recombinant E. coli cells from the fermentation broth 3; and lysing the recombinant E. coli cells to obtain inclusion bodies.

The condition 1 may specifically be as follows: a culture temperature of 37° C.; a dissolved oxygen content (DO) (relative dissolved oxygen content) set to 30%; a pH value of 7.2; culture continues until the fermentation system reaches an OD₆₀₀>25.

The condition 2 may specifically be as follows: a culture temperature of 25° C.; a dissolved oxygen content (DO) (relative dissolved oxygen content) set to 30%; a pH value of 7.2; and culture continues until the OD₆₀₀ of the fermentation system reaches 48.2.

The composition of the fermentation medium may be as follows: each 1 L of the fermentation medium contains 29-31 g of glycerol, 9-11 g of yeast powder, 19-21 g of tryptone, 9-11 g of sodium chloride, 0.9-1.1 mL of defoaming agent, and the remainder is water; with a pH value of 7.2-7.4. Wherein, the pH value of the fermentation medium may be controlled by 30% (volume percentage) ammonia solution.

More specifically, the composition of the fermentation medium may be as follows: each 1 L of the fermentation medium contains 30 g of glycerol, 10 g of yeast powder, 20 g of tryptone, 10 g of sodium chloride, 1 mL of defoaming agent, and the remainder is water; with a pH value of 7.2.

The feed supplement comprises water and solutes, where the solutes and their concentrations may be: 95-105 g/L yeast powder and 190-210 g/L tryptone, specifically 100 g/L yeast powder and 200 g/L tryptone.

The method for collecting the recombinant E. coli cells may be at least one of centrifugation or filtration, specifically centrifugation; the method for lysing the recombinant E. coli cells may be at least one of high-pressure homogenization, freeze-thawing or ultrasonic lysis, specifically high-pressure homogenization.

In step (3), denaturing the inclusion bodies is performed in a denaturing buffer. The pH value of the denaturing buffer is 8.5-9.5; the denaturing buffer comprises a solvent and solutes, where the solvent is water, and the solutes are tris(hydroxymethyl)aminomethane, dithiothreitol and urea; the concentration of tris(hydroxymethyl)aminomethane in the denaturing buffer is 20-50 mM; the concentration of dithiothreitol in the denaturing buffer is 10-50 mM; and the concentration of urea in the denaturing buffer is 7.5-8.5 M.

More specifically, in one embodiment of the present invention, the pH value of the denaturing buffer is 9.0; the denaturing buffer comprises a solvent and solutes, where the solvent is water, and the solutes are tris(hydroxymethyl)aminomethane, dithiothreitol, and urea; the concentration of tris(hydroxymethyl)aminomethane in the denaturing buffer is 20 mM; the concentration of dithiothreitol in the denaturing buffer is 10 mM; and the concentration of urea in the denaturing buffer is 8 M.

The denaturation is carried out at 20-30° C. (room temperature) for 3-5 h. After denaturation, the supernatant is collected by centrifugation to obtain the denatured sample.

During denaturation, the ratio of the inclusion bodies to the denaturing buffer may be specifically 1 g:40 mL.

In step (4), when renaturing the denatured sample, the ratio of the denatured sample to the renaturing buffer may specifically be 1:20 (by volume).

The renaturation is performed at 4° C. for 8-16 h (overnight), such as 10 h.

The recombinant lysine N-terminal protease prepared using the method described above is also within the scope of protection of the present invention.

The invention also provides a set of reagents and a kit for preparing the recombinant lysine N-terminal protease.

The set of reagents provided by the present invention for preparing recombinant lysine N-terminal protease comprises the renaturing buffer; the equilibration buffer and the elution buffer used for the cation exchange chromatography; and the elution buffer used for the gel filtration chromatography.

The kit provided by the present invention for preparing recombinant lysine N-terminal protease comprises the set of reagents and all or part of the following: E. coli competent cells, a prokaryotic expression vector capable of expressing the recombinant lysine N-terminal protease having the amino acid sequence shown in SEQ ID NO. 2 in the sequence listing, IPTG, SP Sepharose Fast Flow or SP Sepharose High Performance, and Superdex 75 Prep Grade.

The kit also contains a readable carrier that records the method for preparing recombinant lysine N-terminal protease described above.

Experiments have demonstrated that the recombinant LysN protease prepared according to the method of the present invention exhibits high enzyme activity and specificity. It is also tolerant to 8 M urea and 1% sodium dodecyl sulfate. The enzyme activity of the recombinant LysN protease is 1433 Azocasein units/mg enzyme; while the enzyme activity of commercial lysine N-terminal protease is 433 Azocasein units/mg enzyme. The preparation method of the present invention has several advantages, including high enzyme activity, good specificity, strong tolerance, and the absence of animal-derived viruses. The production and preparation process is simple and cost-effective, and can be applied to biopharmaceuticals and proteomics research.

### DESCRIPTION OF THE DRAWINGS

FIG. 1 shows the alignment results between the amino acid sequence of SL-LysN after conservative sequence point mutation and the predicted lysine N-terminal protease of Shewanella loihica.

FIG. 2 shows the growth curve of E. coli fermentation. The OD₆₀₀ value at the detection point marked 26.6 corresponds to the OD₆₀₀ value of the fermentation broth at the time of IPTG induction, while the value marked 48.2 represents the OD₆₀₀ value of the fermentation broth at the time of cell collection.

FIG. 3 shows the results of 12% SDS-PAGE electrophoresis analysis of total bacterial cell protein before induction, total bacterial cell protein after induction, supernatant, and inclusion bodies of the BL21-LysN strain.

FIG. 4 shows the chromatogram of cation exchange chromatography. The chromatographic peaks collected by NaCl gradient elution are in the range enclosed by dotted lines.

FIG. 5 shows the chromatogram of gel filtration chromatography. The elution peaks collected by gel chromatography are in the range enclosed by the dotted lines.

FIG. 6 shows the results of polyacrylamide gel (SDS-PAGE) analysis for each purification step.

FIG. 7 shows the base peaks of LC/MS analysis of peptide fragments produced by LysargiNase digestion of purified LysN.

FIG. 8 shows the secondary mass spectrum of the C-terminal peptide of purified LysN.

FIG. 9 shows the secondary mass spectrum of the N-terminal peptide of purified LysN.

FIG. 10 shows the amino acid sequence matching result of purified LysN.

FIG. 11 shows the results of liquid chromatography/mass spectrometry (LC/MS) analysis of the molecular weight of purified LysN.

FIG. 12 shows the results of enzyme activity of purified LysN determined by the azocasein assay.

FIG. 13 shows the enzyme cleavage results of purified LysN on HEK293 total protein at different urea concentrations.

FIG. 14 shows the enzyme cleavage results of purified LysN on HEK293 total protein at different SDS concentrations.

FIG. 15 shows the enzyme cleavage results of purified LysN on HEK293 total protein at different enzyme-to-substrate ratios.

FIG. 16 shows the base peaks of mass spectrometry analysis of a sample digested with purified LysN on HEK293 total protein at a ratio of 1:50.

### DETAILED DESCRIPTION OF THE INVENTION

The following examples are provided to illustrate the invention in detail, but are not intended to limit the scope of the claims. All materials used in the invention are publicly available.
High-density basal fermentation medium: Each 1 L of the fermentation medium contains 10 g of yeast powder, 20 g of tryptone, 30 g of glycerol, 10 g of NaCl, 1 mL of defoaming agent, and the remainder is water; with a pH value of 7.2.
Feed supplement: composed of water and solutes, with the solutes and their concentrations being: 100 g/L yeast powder and 200 g/L tryptone.
Washing buffer for inclusion bodies: composed of solute and solvent, with the solvent being a 20 mM tris(hydroxymethyl)aminomethane-hydrochloride (Tris-HCl) buffer at pH 7.5, and the solute and its concentration being 2 M urea.
Denaturing buffer: composed of a solvent and solutes, with the solvent being water, and the solutes and their concentration being: 20 mM tris(hydroxymethyl)-aminomethane (Tris), 10 mM dithiothreitol (DTT), and 8 M urea; with a pH value of 9.0.
Renaturing buffer: composed of a solvent and solutes, with the solvent being water and the solutes and their concentration being: 20 mM tris(hydroxymethyl)-aminomethane (Tris), 1 mM cystine, 5 mM cysteine, 0.3 M glycine, 0.1 M arginine hydrochloride (Arginine-HCl), and 2.5% glycerol; pH 8.5.
Equilibration buffer used for cation exchange chromatography: composed of a solvent and solutes, with the solvent being water, and the solutes and their concentrations being: 25 mM NaAC, 1 mM EDTA-2Na, pH 4.0.
Elution buffer used for cation exchange chromatography: composed of a solvent and solutes, with the solvent being water, and the solutes and their concentrations being: 25 mM NaAC, 1 mM EDTA-2Na, 1 M NaCl, pH 4.0.
Elution buffer used for gel filtration chromatography: composed of a solvent and solutes, with the solvent being water, and the solutes and their concentrations being: 25 mM NaAC, 1 mM EDTA-2Na, pH 4.0.

### Example 1: Screening and Identification of Gene Encoding Lysine N-Terminal Protease with High Activity, High Specificity, and Strong Tolerance

Through in vitro transcription and translation experiments, apart from G. frondosa, among the seven host strains with high activity of lysine N-terminal protease, two are fungi: Ajellomyces dermatitidis and Verticillium albo-atrum, while the other five are bacteria: Chromobacterium violaceum, Xanthomonas axonopodis, Shewanella loihica, Collimonas fungivorans, and Aeromonas salmonicida. Among these, the in vitro transcription and translation products of the 38.9 kDa predicted protein encoded by the gene YP_001093372.1 (SEQ ID NO. 1) from Shewanella loihica exhibited the most efficient enzymatic cleavage of the substrate protein. SDS-PAGE analysis showed that, except for the electrophoresis front, there were basically no visible protein residues. It had good lysine specificity, making it a good candidate for lysine N-terminal protease.

Sequence alignment analysis was performed on the enzyme proteins with lysine N-terminal protease activity from five bacteria: Chromobacterium violaceum, Xanthomonas axonopodis, Shewanella loihica, Collimonas fungivorans, and Aeromonas salmonicida. After aligning the zinc-binding motif HEXXH and the metal-coordinating motif responsible for catalysis GTXDXXYG, a conservative sequence point mutation study was conducted on the amino acid residues important for enzyme activity and their surrounding sequences (Rawlings et al., Evolutionary families of metallopeptidases. Methods Enzymol, 1995, 248:183-228; Fushimi et al., Aspzincin, a family of metalloendopeptidases with a new zinc-binding motif.

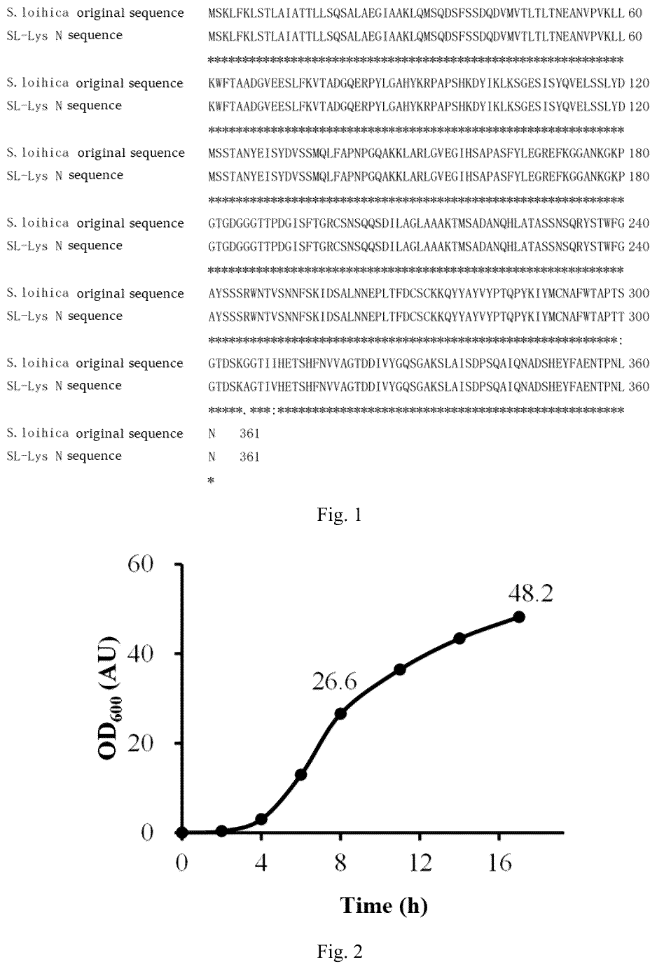

Identification of new zinc-binding sites (His (128), His (132), and Asp (164)) and three catalytically crucial residues (Glu (129), Asp (143), and Tyr (106)) of deuterolysin from Aspergillus oryzae by site-directed mutagenesis. J Biol Chem, 1999, 274:24195-24201), resulting in the lysine N-terminal protease sequence, named SL-LysN. The sequence alignment results between this sequence and the predicted lysine N-terminal protease sequence from the Shewanella loihica genome are shown in FIG. 1. The mature protein form of this protein is unknown and is also determined by this application.

### Example 2: Construction, Fermentation, and Inclusion Body Preparation of Genetically Engineered Recombinant E. coli Expressing a Lysine N-Terminal Protease with High Activity, High Specificity, and Strong Tolerance

### 1. Optimization of the Gene Encoding Lysine N-Terminal Protease (LysN)

The gene encoding SL-LysN (SEQ ID NO. 1) was optimized by replacing its codons with those preferred by E. coli (high-frequency use), without altering the amino acid sequence of lysine N-terminal protease (positions 11-348 of SEQ ID NO. 2).

The optimization also includes other modifications to the gene sequence of lysine N-terminal protease to suit expression in E. coli, ultimately yielding the optimized gene sequence encoding LysN shown in SEQ ID NO. 3. We also introduced a 6×His tag at its N-terminus.

### 2. Construction of Recombinant Expression Vector

The DNA sequence shown in SEQ ID NO. 3 was inserted into the Nco I/Xho I restriction enzyme sites of the pET28a vector (Novagen, catalog number 69864-3CN) yielding the recombinant expression vector for lysine N-terminal protease, named pET-LysN. Further DNA sequencing verification confirmed the correct construction of the vector.

### 3. Plasmid Transformation

Using chemical transformation, the pET-LysN vector constructed in step 2 was introduced into E. coli BL21 (DE3) competent cells, yielding a recombinant strain containing the recombinant LysN gene. This strain was named recombinant E. coli BL21-LysN (hereinafter referred to as the BL21-LysN strain).

### 4. Fermentation Seed Preparation

The BL21-LysN strain constructed in Example 2 was activated on solid LB plates (containing 50 μg/mL kanamycin) to obtain a single colony of the BL21-LysN strain. The single clone was inoculated into 25 mL of LB liquid medium (containing 50 μg/mL kanamycin) and cultured at 37° C. in a shaking incubator. When the OD₆₀₀ of the LB liquid medium reached 1-1.5, the fermentation seed solution was obtained.

### 5. Fermentation of the BL21-LysN Strain

Seed inoculation was performed in a 5 L fully automated fermenter (Shanghai Baoxing Bio, 5JG) containing 1.8 L of high-density fermentation basal medium that had been autoclaved at 121° C. The OD₆₀₀ of the culture system after inoculation was 0.01. We investigated the effect of fermentation temperature on SL-LysN expression levels under 1 mM IPTG induction conditions, as shown in Table 1. The results showed that under the conditions of a stirring speed of 500 rpm, an air flow rate of 6 L/min, a pH of 7.2, and under the control of 30% ammonia solution during fermentation, the expression level of LysN was the highest at a fermentation temperature of 25° C.

TABLE 1
Effect of fermentation temperature on LysN expression levels

| Fermentation temperature | 15° C. | 20° C. | 25° C. | 30° C. |
|---|---|---|---|---|
| Percentage of target protein | 15.3% | 16.7% | 20.6% | 13.7% |

Furthermore, we investigated the effect of IPTG induction concentration on LysN expression levels, with the results shown in Table 2. The results showed that under the fermentation conditions of a stirring speed of 500 rpm, an air flow rate of 6 L/min, a pH of 7.2, and under the control of 30% ammonia solution during fermentation, and a fermentation temperature of 25° C., the LysN expression level was the highest under 1 mM IPTG induction.

TABLE 2
Effect of IPTG induction concentration on LysN expression levels

| IPTG concentration | 0.1 mM | 0.4 mM | 0.7 mM | 1.0 mM |
|---|---|---|---|---|
| Percentage of target protein | 12.2% | 14.3% | 18.5% | 20.6% |

Under optimized fermentation conditions (an IPTG induction concentration of 1 mM, a culture temperature of 25° C., a stirring speed of 500 rpm, an air flow rate of 6 L/min, a pH of 7.2, and under the control of 30% ammonia solution (volume percentage concentration) during fermentation), the fermentation system was cultured until the OD₆₀₀ reached 48.2, yielding fermentation broth 2. The growth curve of E. coli fermentation is shown in FIG. 2.

The bacterial cells were collected after centrifugation at 4° C. for 30 min.

### 6. Preparation of LysN Inclusion Bodies

The supernatant was discarded, and 1×PBS buffer was added to the bacterial cells at a ratio of 1 g:10 mL (w/v). The mixture was homogenized using a high-speed tissue homogenizer and then lysed using a high-pressure homogenization method. The high-pressure homogenization pressure was set to 800 bar, and the cells were lysed three times consecutively. The precipitate was collected by centrifugation at 4000 rpm and 4° C. for 30 min, yielding the inclusion bodies of the BL21-LysN strain, designated as Inclusion body 1.

Inclusion body 1 was washed with PBS+2 M urea and centrifuged under the same conditions to collect the inclusion bodies, yielding Inclusion body 2.

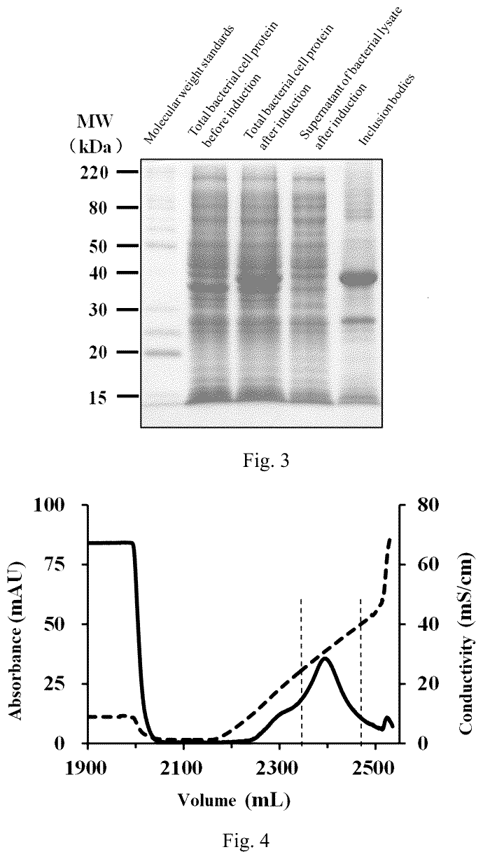

Total cellular protein before induction, total cellular protein after induction, supernatant, and inclusion bodies of the BL21-LysN strain were analyzed by 12% SDS-PAGE electrophoresis, with results shown in FIG. 3. Compared to the total cellular protein of the BL21-LysN strain before induction, a new protein band of approximately 37.5 kDa was detected in both the total cellular protein after induction and the washed inclusion bodies of the BL21-LysN strain. The molecular weight matches the expected theoretical molecular weight of 37,585.49 Da, suggesting that it may be the LysN target protein, indicating that the LysN target protein is primarily expressed in the form of inclusion bodies.

### Example 3: Activation and Purification of Recombinant Lysine N-Terminal Protease

### 1. Denaturation of Inclusion Body

The inclusion bodies were dissolved in the denaturing buffer at a ratio of 1:40 (by mass-to-volume ratio, g/mL), denatured at room temperature for 4 hours, and centrifuged at 17,000 rpm to collect the supernatant, which served as the denatured sample solution.

### 2. Renaturing of Protein

The denatured sample obtained in step 1 was added to the renaturing buffer at a ratio of 1:20 (by volume), renatured overnight at 4° C., thus obtaining the renaturing sample.

### 3. Activation of Lysine N-Terminal Protease

The effects of different temperature conditions on the proenzyme activation and yield of lysine N-terminal protease were investigated. A zinc sulfate solution was added to the renatured samples obtained in above step 2 to a final concentration of 1 mM. The samples were then activated for 2 hours at 4° C., 8° C., 12° C., 16° C., 20° C., 24° C., 28° C., and 32° C. The relationship between the amount of newly generated lysine N-terminal protease and activation temperature is shown in Table 3. The results showed that the optimal activation condition was 16° C., yielding the highest amount of newly generated lysine N-terminal protease.

TABLE 3
Effect of activation temperature on LysN

| Activation | | | | | | | | |
|---|---|---|---|---|---|---|---|---|
| temperature | 4 | 8 | 12 | 16 | 20 | 24 | 28 | 32 |
| LysN yield | 0.61 | 0.65 | 0.75 | 1.08 | 0.91 | 0.82 | 0.53 | 0.28 |
| (mg/g | | | | | | | | |
| inclusion | | | | | | | | |
| body) | | | | | | | | |

As shown in FIG. 6, the newly generated LysN has a molecular weight of about 20 kDa.

### 4. Purification of the Activated Protein

The activated sample obtained in above step 3 was divided into two portions. One portion was adjusted to pH 4.0 and sodium acetate-acetic acid buffer (pH 4.0) was added to a final concentration of 25 mM. The mixture was then centrifuged at 8000 rpm for 30 min to collect the supernatant. The obtained supernatant was subjected to cation exchange chromatography using an SP Sepharose Fast Flow column with a column volume of 60 mL. Another sample was adjusted to pH 8.0 and centrifuged at 8000 rpm for 30 min to collect the supernatant. The supernatant was subjected to anion exchange chromatography using a Q Sepharose High Performance column with a column volume of 60 mL. The results are shown in Table 4.

TABLE 4
Effect of different chromatographic media on LysN purification

| Chromatography | Cation exchange | Anion exchange |
|---|---|---|
| method | chromatography | chromatography |
| Purification | LysN was successfully | No LysN was detected |
| results | enriched in the elution | in the elution peak, |
| | peak, with a purity of | but LysN was detected |
| | 60%. | in the flow-through. |

The results showed that only cation exchange chromatography could effectively enrich LysN.

The chromatogram of cation exchange chromatography is shown in FIG. 4. The SDS-PAGE electrophoresis results are shown in FIG. 6, indicating that the obtained LysN has a molecular weight of about 20 kDa.

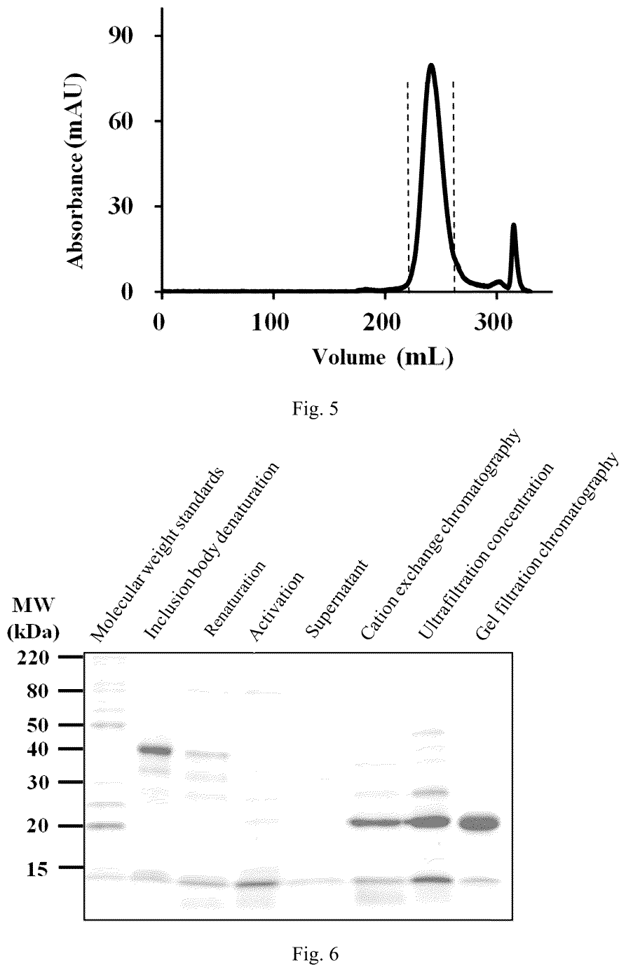

The target protein was concentrated using a 10 kD centrifugal concentration tube from Merck-Millipore and separated by molecular sieving using a “XK 26/600 column” from GE Healthcare. Following the instructions for GE Healthcare's “XK 26/600 column,” pure LysN was obtained. The molecular sieve gel filtration chromatography is shown in FIG. 5.

The collected elution peaks were analyzed by SDS-PAGE, with the results shown in FIG. 6. The molecular weight was about 20 kDa, consistent with the previous results, and no visible impurity bands were observed.

### Example 4: Identification of the Primary Structure Amino Acid Sequence of Recombinant Lysine N-Terminal Protease

LC-MS/MS analysis was performed on the purified recombinant lysine N-terminal protease product obtained after gel filtration chromatography.

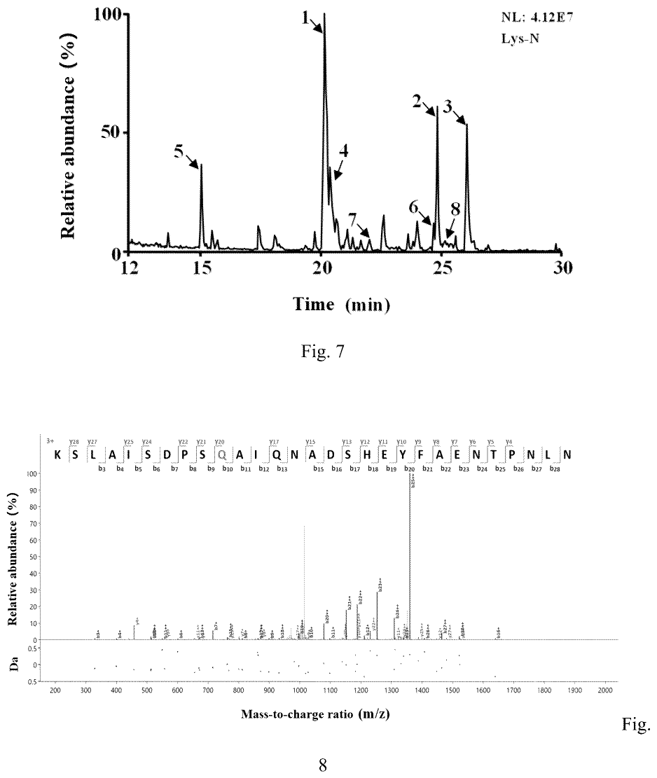

The LC-MS/MS base peak results of LysN digested by LysargiNase are shown in FIG. 7. A total of 8 LysN peptide fragments were identified, with the sequence results shown in Table 5. Among them, the secondary spectrum of the peptide fragment KSLAISDPSQAIQNADSHEYFAENTPNLN is shown in FIG. 8, with good subion matching and unambiguous identification. This peptide fragment is positioned immediately at the carboxy-terminal end of the recombinant target protein pro-LysN coding sequence, indicating that the mature LysN terminates at this peptide fragment.

TABLE 5
Sequences of eight LysN peptide
fragments identified

| No. | Amino acid sequence |
|---|---|
| 1 | KGKPGTGDGGGTTPDGISFTG |
| 2 | KSLAISDPSQAIQNADSHEYFAENTPNLN |
| 3 | RCSNSQQSDILAGLAAA |
| 4 | RWNTVSNNFS |
| 5 | KTMSADANQHLATASSNSQ |
| 6 | KIDSALNNEPLTFDCSC |
| 7 | KKQYYAYVYPTQPY |
| 8 | RYSTWFGAYSSS |

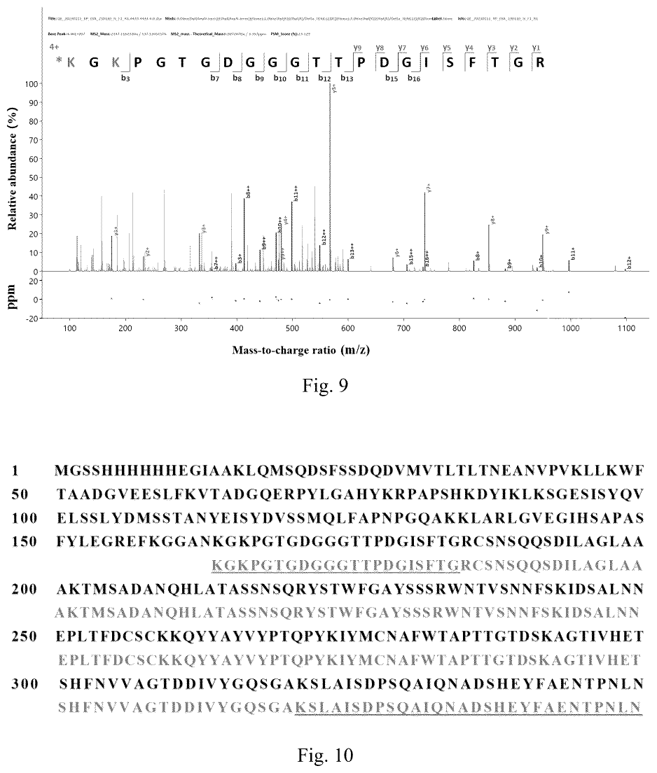

To determine the amino acids of mature LysN, we labeled the purified target protein with dimethyl and then digested it with Trypsin. Digestion products were analyzed by LC-MS/MS, and the dimethyl-labeled peptide fragment K*GKPGTGDGGGTTPDGISFTG (where K* represents the dimethyl-labeled lysine residue) was identified. Its secondary spectrum is shown in FIG. 9, with good ion matching and unambiguous identification, indicating that the N-terminus of mature LysN begins at this lysine residue.

Based on the results of the carboxy-terminal and amino-terminal analysis of purified mature LysN identified by proteomics, the amino acid sequence of LysN was determined, as shown in FIG. 10. The theoretical molecular weight is 19,787.19 Da, which is consistent with the experimental molecular weight of about 20 kDa obtained from electrophoresis of mature LysN in FIG. 6.

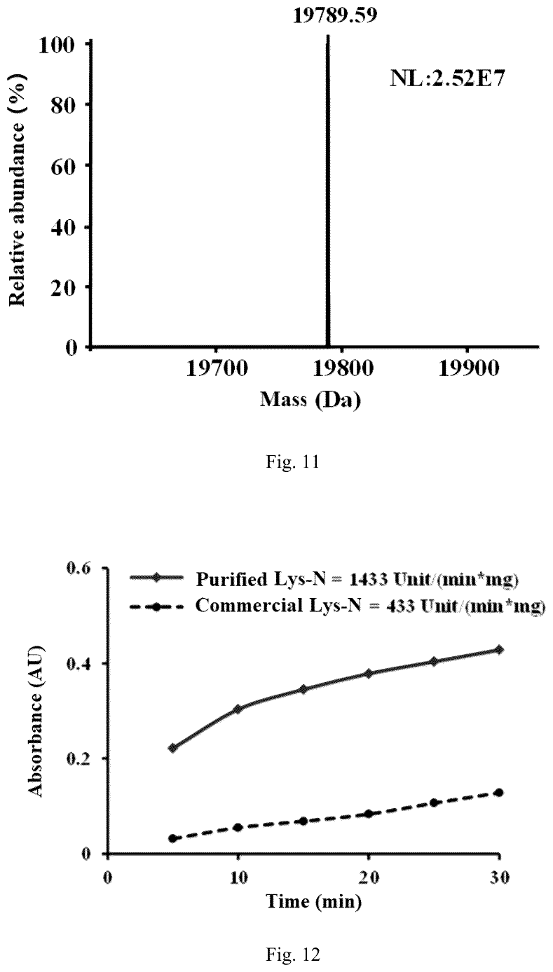

To determine the exact molecular weight of the purified mature LysN, we performed LC-MS/MS analysis on the recombinant lysine N-terminal protease prepared. The liquid chromatograph was a RIGOL L-3000, with a mobile phase of 20 mM ammonium acetate and a flow rate of 0.2 mL/min. The mass spectrometer was an Exactive Plus EMR, with a mass resolution set to 35,000. The results showed a single chromatographic peak with a molecular weight of 19,789.59 Da, consistent with the theoretical molecular weight of 19,787.19 Da. The difference between the measured and theoretical molecular weights was within the instrument's detection error range (±150 ppm), further confirming that the identified mature LysN has correct carboxy-terminal and amino-terminal sequences (FIG. 11).

These results confirm that we have obtained a novel LysN with an unambiguously identified primary structure sequence.

### Example 5: Detection of the Activity of Recombinant Lysine N-Terminal Protease (LysN) Using the Azocasein Method

The recombinant lysine N-terminal protease (LysN) solution prepared in Example 1 was diluted to a concentration of 0.015 mg/mL using a 50 mM zinc acetate solution. Similarly, the commercial LysN solution was diluted to a concentration of 0.015 mg/mL using a 50 mM zinc acetate solution.

0.1 mL of the recombinant lysine N-terminal protease (LysN) solution (test enzyme solution) with a concentration of 0.015 mg/mL was added to 1.4 mL of azocasein solution with a concentration of 0.2% (the solution was composed of a solvent and a solute. The solvent was a 0.1 M glycine-sodium hydroxide solution with a pH of 10.0; the solute was azocasein at a concentration of 0.2% (by mass-to-volume ratio)), and incubated at 37° C. for 30 min. 0.1 mL of the commercial LysN solution (test enzyme solution) with a concentration of 0.015 mg/mL was added to 1.4 mL of azocasein solution with a concentration of 0.2% (as above), and incubated at 37° C. for 30 min. The blank control was a mixture of 0.1 mL of 50 mM zinc acetate solution and 1.4 mL of 0.2% azocasein solution (as above).

At 5 min, 10 min, 15 min, 20 min, 25 min and 30 min, 0.22 mL of the recombinant lysine N-terminal protease (LysN) enzyme reaction solution, the commercial Lys-N enzyme reaction solution, or the blank control mixture was added with 0.22 mL of 5% trichloroacetic acid to terminate the reaction, respectively, followed by standing at 25° C. for 20 min and then centrifuged at 12,000 rpm using a horizontal rotor for 10 min. 0.4 mL of supernatant was added with 0.6 mL of 0.2 M NaOH for chromogenic development. UV absorption at 440 nm was measured using an Agilent Cary 60 UV-Vis spectrophotometer.

LysN can hydrolyze azocasein into peptides. Unreacted azocasein and LysN were precipitated with trichloroacetic acid, and the hydrolyzed peptides exhibited maximum UV absorption peak at 440 nm. As the peptide product amount increased, the UV absorption showed a linear increase. By measuring the UV absorbance value OD₄₄₀, the change in UV absorption per unit time can be calculated, thereby determining the enzyme activity of LysN. The calculation formula is as follows:

$$Azocasein Units / mg enzyme = \frac{( Δ {OD}_{440} / min Test - Δ {OD}_{440} / min Blank )}{( 0.01 ) ( 0.001 ) ( 0.2 )}$$

where, “Test” refers to the recombinant lysine N-terminal protease (LysN) enzyme reaction solution or the commercial LysN enzyme reaction solution, “Blank” refers to the blank control.

The LysN enzyme activity unit was defined as the change in OD₄₄₀ per unit time, using azocasein as the substrate under reaction conditions of pH 10.0 and 37° C., with a total volume of 1.5 mL, and a 1 cm light path. An increase of 0.01 in OD₄₄₀ per minute was defined as one Azocasein unit.

The results showed that the enzyme activities of both recombinant lysine N-terminal protease (LysN) and commercial LysN increased linearly within 30 min. The enzyme activity of the recombinant lysine N-terminal protease (LysN) was more than three times that of commercial LysN. The specific results are shown in FIG. 12.

The enzyme activity of the recombinant lysine N-terminal protease (SL-LysN) prepared in Examples 1-3 was 1,433 Azocasein units/mg enzyme, while that of the commercial LysN was 433 Azocasein units/mg enzyme. The enzyme activity of the recombinant lysine N-terminal protease (SL-LysN) of the present invention was more than three times higher than that of commercial Lys-N, demonstrating significantly improved activity.

### Example 6: Activity Measurement of Recombinant Lysine N-Terminal Protease (LysN) Based on Large-Scale Genomics

Human embryonic kidney 293 cells were resuspended in PBS, sonicated, and centrifuged to obtain total bacterial cell protein. The protein was precipitated with pre-cooled acetone at a 1:9 ratio, then resuspended in resuspension buffer (formulation: solvent: water, solutes and their concentrations: 50 mM ammonium bicarbonate and 8 M urea). The protein concentration was 4 mg/mL. DDT was added to a final concentration of 10 mM, and the mixture was incubated at 37° C. for 45 min. After cooling the sample to room temperature, iodacetamide was added to a final concentration of 20 mM, and alkylation was performed in the dark for 30 min. After alkylation, the solution was buffer-exchanged using a 3 kD ultrafiltration tube to remove DTT and iodacetamide. The sample was then diluted with buffer (formulation: solvent: water; solutes and their concentrations: 50 mM ammonium bicarbonate, 1 mM zinc acetate, and different concentrations of urea or sodium dodecyl sulfate) to prepare seven human embryonic kidney cell 293 total protein samples. Four samples of human embryonic kidney cell 293 total protein contained 1 mg/mL protein, 50 mM ammonium bicarbonate, 1 mM zinc acetate, and urea concentrations of 2 M, 4 M, 6 M, and 8 M, respectively, for later use. The remaining three samples of human embryonic kidney cell 293 total protein contained 1 mg/mL protein, 50 mM ammonium bicarbonate, 1 mM zinc acetate, and sodium dodecyl sulfate concentrations of 0.25%, 0.5%, and 1%, respectively, for later use.

The total protein samples of human embryonic kidney cells 293 were digested using the purified recombinant lysine N-terminal protease (SL-LysN) prepared according to the present invention. The specific steps were as follows:
1. The purified recombinant lysine N-terminal protease (SL-LysN) was added to the human embryonic kidney cell 293 total protein samples (with urea concentrations of 2M, 4M, 6M, and 8M) prepared in the above step at a ratio of 1:25 (by weight). Enzymatic digestion was carried out at 37° C. for 2 hours, followed by SDS-PAGE analysis.

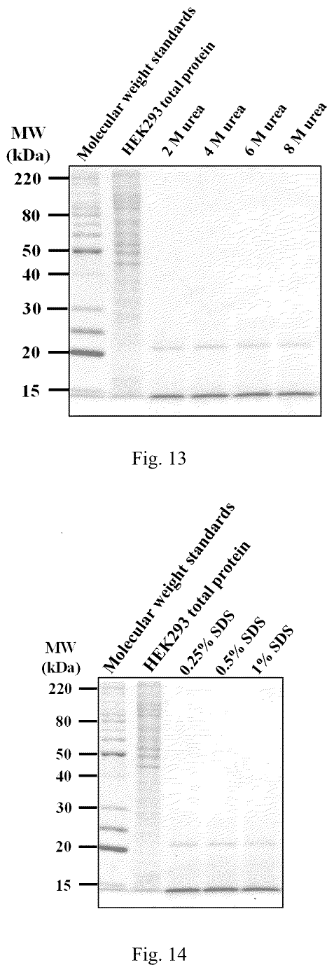

The results are shown in FIG. 13. Based on the electrophoresis results, the protease completely digested the human embryonic kidney cell 293 total protein under urea concentrations ranging from 2 to 8 M.
2. The purified recombinant lysine N-terminal protease (SL-LysN) was added to the human embryonic kidney cell 293 total protein samples (with 0.25%, 0.5%, and 1% sodium dodecyl sulfate concentration, respectively) prepared in the above step at a ratio of 1:25 (by weight). Enzymatic digestion was carried out at 37° C. for 2 hours, followed by SDS-PAGE analysis.

The results are shown in FIG. 14. Based on the electrophoresis results, the protease completely digested the human embryonic kidney cell 293 total protein under sodium dodecyl sulfate concentrations ranging from 0.25% to 1%.
3. The purified recombinant lysine N-terminal protease (SL-LysN) was added to the human embryonic kidney cell 293 total protein samples (with 2M urea concentration) prepared in the above step at a ratio of 1:(50-1000) (by weight). Enzymatic digestion was carried out at 37° C. for 2 hours, followed by SDS-PAGE analysis.

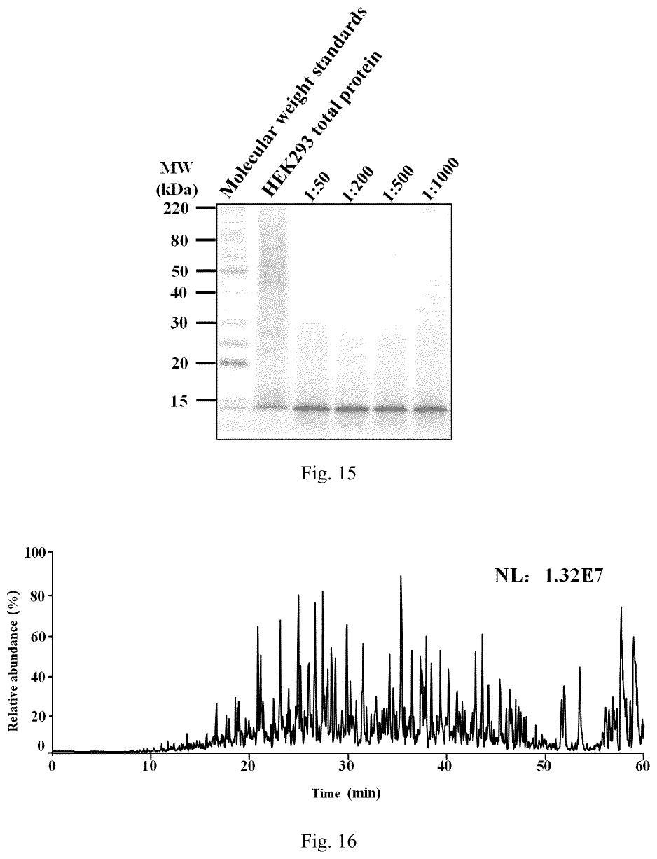

The results are shown in FIG. 15. Based on the electrophoresis results, the protease completely digested the human embryonic kidney cell 293 total protein under the digestion conditions used in the analysis.
4. The purified recombinant lysine N-terminal protease (SL-LysN) was added to the human embryonic kidney cell 293 total protein samples (with 2 M urea concentration) prepared in the above step at a ratio of 1:50 (by weight). Enzymatic digestion was carried out overnight (14 hours). The total amount of human embryonic kidney cell 293 total proteins in the system was 20 μg. After digestion, the reaction was terminated by adding formic acid to a final concentration of 0.2%, followed by C₁₈StageTip desalting for mass spectrometry analysis.

Mass spectrometry instrumentation and parameters: Ultra-high-pressure liquid chromatography system (Waters Corporation, Milford, MA, USA); mass spectrometry system (LTQ Orbitrap Velos, ThermoFisherScientific, San Jose, CA, USA). Samples were subjected to a 60-minute liquid chromatography gradient separation on a custom-made reverse-phase column (75 μm×15 cm, 3 μm, 200 Å), where mobile phase A: 0.1% FA, 100% ultra-pure water, mobile phase B: 0.1% FA, 100% ACN (acetonitrile); flow rate: 400 nL/min. Eluted peptides were analyzed by mass spectrometry with a scan range of 300-1600 m/z. The top 20 most abundant ions were selected for CID (Collision-induced Dissociation) in the LTQ (linear ion trap). The primary spectrum resolution and secondary spectrum resolution were 30,000 and 7,500, respectively (at 400 m/z). For each scan, the maximum ion number accumulated within 25 ms was 5,000. The dynamic exclusion time for parent ions was 30 s.

The obtained mass spectrometry data were analyzed using MaxQuant (version 5.5.1).

The mass spectrometry results are shown in FIG. 16 and Table 6. The results showed that among the identified peptide fragments, those with lysine as the N-terminal accounted for over 94% of the total peptide fragments. This demonstrates that the recombinant SL-LysN protein prepared in Example 1 of the present invention exhibits extremely high cleavage specificity, it can be used to prepare proteomics sample.

TABLE 6
Detection of specificity of the recombinant proteases
based on human embryonic kidney cell 293 total protein

| | Protein | Peptide | Lysine as the | Lysine |
|---|---|---|---|---|
| | No. | fragment No. | N-terminus | specificity |
| Experiment 1 | 1280 | 3772 | 3575 | 94.78% |
| Experiment 2 | 1275 | 3714 | 3522 | 94.83% |
| Experiment 3 | 1372 | 4058 | 3829 | 94.36% |

1. An isolated or recombinant lysine N-terminal protease, wherein the recombinant lysine N-terminal protease has an amino acid sequence shown in SEQ ID NO. 2.

2. An isolated or recombinant nucleic acid encoding the recombinant lysine N-terminal protease of claim 1, wherein the nucleic acid has a nucleotide sequence shown in SEQ ID NO. 3.

3. An expression vector, containing the nucleic acid of claim 2.

4. The expression vector of claim 3, wherein the expression vector is a pET28a vector.

5. A host cell, containing the expression vector of claim 3.

6. The host cell of claim 5, wherein the host cell is an E. coli cell.

7. A method for preparing the recombinant lysine N-terminal protease of claim 1, comprising the following steps:
(a) operably linking a gene shown in SEQ ID NO. 3 that encodes the recombinant lysine N-terminal protease to an expression vector, and introducing the expression vector into a host cell;
(b) culturing the host cell under expression conditions to express the recombinant lysine N-terminal protease; and
(c) isolating and purifying the recombinant lysine N-terminal protease from (b).

8. The method of claim 7, wherein the expression vector is a pET28a vector and the host cell is an E. coli cell.

9. The method of claim 8, comprising the following steps:
(1) introducing the gene shown in SEQ ID NO. 3 that encodes the recombinant lysine N-terminal protease into recipient E. coli cells to obtain recombinant E. coli cells;
(2) inducing expression of the recombinant E. coli cells, collecting the recombinant E. coli cells; and lysing the recombinant E. coli cells to obtain inclusion bodies;
(3) denaturing the inclusion bodies to obtain a denatured sample;
(4) renaturing the denatured sample to obtain a renatured sample;
(5) activating the renatured sample to obtain crude recombinant lysine N-terminal protease with activity; and
(6) purifying the crude recombinant lysine N-terminal protease to obtain purified recombinant lysine N-terminal protease;
wherein in step (2), the induction expression in the recombinant E. coli cells comprises adding IPTG (Isopropyl β-D-1-thiogalactopyranoside) to the culture system containing the recombinant E. coli to a final concentration of 0.7-1.2 mM, followed by induction at 25° C. for 9 hours
in step (3), the denaturation is performed in a denaturing buffer, which has a pH value of 8.5-9.5; the denaturing buffer comprises a solvent and solutes, where the solvent is water, and the solutes are tris(hydroxymethyl)aminomethane, dithiothreitol, and urea; the concentration of tris(hydroxymethyl)aminomethane in the denaturing buffer is 20-50 mM, the concentration of dithiothreitol in the denaturing buffer is 10-20 mM, and the concentration of urea in the denaturing buffer is 7.5-8.5 M;
in step (4), the renaturation is performed in a renaturing buffer, which has a pH value of 8.0-10.0; the renaturing buffer comprises a solvent and solutes, where the solvent is water, and the solutes are tris(hydroxymethyl)aminomethane, cystine, cysteine, glycine, arginine hydrochloride, and glycerol; the concentration of tris(hydroxymethyl)aminomethane in the renaturing buffer is 10-50 mM, the concentration of cystine in the renaturing buffer is 0.5-1.5 mM, the concentration of cysteine in the renaturing buffer is 3-5 mM, the concentration of glycine in the renaturing buffer is 0.1-0.4 M, the concentration of arginine hydrochloride in the renaturing buffer is 0.1-0.2 M, and the concentration of glycerol in the renaturing buffer is 2-5%;
in step (5), the activation comprises adding ZnSO₄ to the renatured sample to a concentration of 1 mM, followed by standing at 16° C. for 1-4 hours; then adding ethylenediaminetetraacetic acid disodium to a concentration of 2 mM to terminate the activation, and adjusting the pH of the sample to 4.0-5.0;
in step (6), the purification comprises sequentially performing cation exchange chromatography, ultrafiltration concentration, and gel filtration chromatography;
wherein the cation exchange chromatography uses a high flow rate agarose gel or high resolution agarose gel as the chromatographic medium; the ultrafiltration concentration is an ultrafiltration concentration with a molecular weight cut-off of 10 kD.

10. The method of claim 9, wherein: in step (2), the induction expression is carried out under the following conditions: a stirring speed of 500 rpm, an air flow rate of 6 L/min, a pH value of 7.2, and under the control of 30% ammonia solution during fermentation, a fermentation temperature of 25° C., and the IPTG concentration is 1 mM;
in step (5), the activation temperature is 16° C.

11. A recombinant lysine N-terminal proteinase prepared by the method of claim 7.


SEQUENCE LISTING (3 sequences)

SEQ ID NO: 1 - DNA, 1086 bp, unidentified
  source 1..1086: mol_type = genomic DNA
atgagcaagctattcaaattatcaacactcgctattgccaccaccttactgagtcaatcagccctcgccgaagggatcgccgctaaactgcagatgagccaagacagcttcagcagcgatcaggacgtgatggtcaccctaaccctgaccaacgaagcaaacgttccggtgaaactactcaagtggtttactgcggcagacggtgttgaagagtcgctatttaaagttaccgctgatggtcaagagcgcccttaccttggtgcccactacaagcgtccagcaccaagccataaagactacatcaagctaaaatccggtgagagcatctcttatcaggtagagctatcgtcactgtatgacatgtcatccacggctaactatgagatcagctatgatgtcagctctatgcagctgtttgcgccaaacccaggtcaggctaagaaactggctcgccttggcgtagaaggcatacactctgccccagcgagcttctacctggaaggccgcgaatttaaaggcggcgctaacaagggcaagccaggcaccggcgatggtggcggcaccacacctgacggcatctctttcaccggtcgttgcagcaactcacagcagagtgatatccttgccggcctagccgcggctaagacaatgtcagccgacgctaaccagcacctggccaccgccagcagcaactcacagcgttacagcacctggttcggcgcttacagctcgtctcgctggaacacggtaagcaacaacttcagcaagatcgatagcgcgctcaacaacgagccactgaccttcgactgtagctgtaagaagcaatactacgcttatgtttacccaactcagccatacaagatctacatgtgtaatgccttctggacagccccaacgtcgggtactgactctaagggcggcaccatcatccatgaaaccagccacttcaacgtagtagcgggcactgatgatattgtttacggtcagagcggtgctaagtcactggcgatcagcgatccttcacaggcgattcaaaatgctgacagccatgaatattttgctgaaaacacgcctaacttaaactaa

SEQ ID NO: 2 - protein, 348 aa, synthetic construct
MGSSHHHHHHEGIAAKLQMSQDSFSSDQDVMVTLTLTNEANVPVKLLKWFTAADGVEESLFKVTADGQERPYLGAHYKRPAPSHKDYIKLKSGESISYQVELSSLYDMSSTANYEISYDVSSMQLFAPNPGQAKKLARLGVEGIHSAPASFYLEGREFKGGANKGKPGTGDGGGTTPDGISFTGRCSNSQQSDILAGLAAAKTMSADANQHLATASSNSQRYSTWFGAYSSSRWNTVSNNFSKIDSALNNEPLTFDCSCKKQYYAYVYPTQPYKIYMCNAFWTAPTTGTDSKAGTIVHETSHFNVVAGTDDIVYGQSGAKSLAISDPSQAIQNADSHEYFAENTPNLN

SEQ ID NO: 3 - DNA, 1047 bp, synthetic construct
  source 1..1047: mol_type = other DNA
atgggttcctctcaccatcatcatcaccacgaaggtattgcagcaaaactgcagatgagccaggatagctttagtagcgatcaggatgtgatggtgacactgacactgacaaatgaagcaaatgtgccggtgaaactgctgaaatggtttacagcggcagatggcgtagaagaaagcctgtttaaagttacagcagatggccaggaacgtccgtatctgggtgcacattataaacgtccggcgcctagtcataaagattatattaaactgaaaagcggcgaaagtattagttatcaggttgaactgagcagtctgtatgatatgtctagtaccgccaattatgaaattagctatgatgtttcaagcatgcagctgtttgcaccaaatccgggtcaggcaaaaaaactggcacgtctgggtgttgaaggtattcatagcgcaccggcaagcttttatctggaaggtcgtgaatttaaaggtggtgcaaataaaggtaaaccgggtaccggtgatggtggtggtaccaccccggatggtattagctttaccggtcgttgtagcaatagccagcagagcgatattctggcaggtctggcagcagcaaaaaccatgagcgcagatgcaaatcagcatctggcaaccgcaagcagcaatagccagcgttatagcacctggtttggtgcatatagcagcagccgttggaataccgttagcaataattttagcaaaattgatagcgcactgaataatgaaccgctgacctttgattgtagctgtaaaaaacagtattatgcatacgtttatccgacccagccgtataaaatttatatgtgtaatgcattttggaccgcaccgaccaccggtaccgatagcaaagcaggtaccattgttcatgaaaccagccattttaatgttgttgcaggtaccgatgatattgtttatggtcagagcggtgcaaaaagcctggcaattagcgatccgagccaggcaattcagaatgcagatagccatgaatattttgcagaaaataccccgaatctgaattaa